(19) United States
(12) Patent Application Publication
(10) Pub. No.: US 2009/0124657 A1
(43) Pub. Date: May 14, 2009

(54) PHARMACEUTICAL COMPOSITIONS COMPRISING MONTELUKAST

(76) Inventors: Ramesh Kappala, Nalgonda (IN); Basety Srikanth, Hyderabad (IN); Rahul Sudhakar Gawande, Nagpur (IN); Subhash Pandurang Gore, Solapur (IN); Irukulla Srinivas, Hyderabad (IN); Indu Bhushan, Hyderabad (IN); Mailatur Sivaraman Mohan, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/191,611

(22) Filed: Aug. 14, 2008

Related U.S. Application Data
(60) Provisional application No. 60/985,485, filed on Nov. 5, 2007.

(30) Foreign Application Priority Data
Aug. 14, 2007 (IN) 1818/CHE/2007

(51) Int. Cl. A61K 31/47; A61P 11/06

(52) U.S. Cl. 514/311

(57) ABSTRACT

Pharmaceutical compositions comprising montelukast or pharmaceutically acceptable salts, solvates, polymorphs, enantiomers or mixtures thereof.

### INTRODUCTION

The present invention relates to pharmaceutical compositions comprising montelukast, including or pharmaceutically acceptable salts, solvates, polymorphs, enantiomers or mixtures thereof. The invention also relates to processes for preparing the compositions and their methods of use.

Montelukast is described chemically as [R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropaneacetic acid (hereinafter referred to by its adopted name “montelukast”) and is structurally represented by Formula I. The pharmacologically active enantiomer of racemic montelukast is the R-enantiomer.

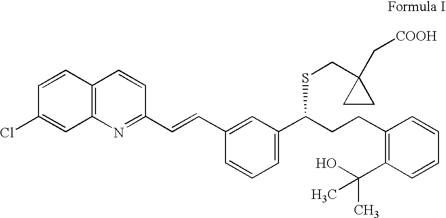

The sodium salt, called montelukast sodium, is a hygroscopic, optically active, and white to off-white powder. The empirical formula for the compound is C₃₅H₃₅ClNNaO₃S, and its molecular weight is 608.18. Montelukast sodium is freely soluble in ethanol, methanol and water and practically insoluble in acetonitrile.

Montelukast is a selective and orally active leukotriene receptor antagonist that inhibits the cysteinyl leukotriene CysLT₁ receptor and is useful in the treatment of asthma as well as other conditions mediated by leukotrienes, such as inflammation and allergies.

Montelukast sodium is marketed in the form of film coated tablets (10.4 mg montelukast sodium, 10 mg montelukast equivalent), chewing tablets (4 and 5 mg montelukast equivalent), and oral granules (5 mg montelukast equivalent), as SINGULAIR®.

It has been reported that montelukast sodium (SINGULAIR®) is indicated for the prophylaxis and chronic treatment of asthma in adults and pediatric patients 12 months of age and older and for the relief of symptoms of allergic rhinitis (seasonal allergic rhinitis in adults and pediatric patients 2 years of age and older, and perennial allergic rhinitis in adults and pediatric patients 6 months and older).

Montelukast, being a hygroscopic molecule, is sensitive to various conditions such as thermal stress, oxidative stress, base hydrolysis and acid hydrolysis, photo degradation, and water hydrolysis leading to formation of impurities.

Among the various impurities of montelukast, mok-3 sulphoxide and styrene impurity (both defined subsequently herein) are the major known impurities. Other known impurities include quid-8, mok-3 keto, mok-1-nitrile, saturated analog, montelukast deschloro impurities and an S-isomer (all defined subsequently).

Regulatory authorities worldwide require that the levels of the impurities should be maintained below the lowest possible levels in the composition. Hence there is a need for stabilized compositions comprising montelukast or its salts.

The present application provides stable pharmaceutical compositions comprising montelukast or its salts.

### SUMMARY

One embodiment of the present invention relates to pharmaceutical compositions comprising montelukast or its pharmaceutically acceptable salts, solvates, polymorphs, enantiomers or mixtures thereof.

In another embodiment, the present invention provides a process for the preparation of pharmaceutical compositions of montelukast or its pharmaceutically acceptable salts.

Another embodiment of the present invention includes stable pharmaceutical compositions comprising montelukast or its pharmaceutically acceptable salts.

In an embodiment, the present invention includes stable pharmaceutical compositions montelukast or its salts, wherein the compositions are stabilized by maintaining equilibrium relative humidity (ERH) of the compositions less than about 25%, 20%, or 15%.

In another embodiment, the present invention includes the pharmaceutical compositions comprising montelukast or its salts, wherein ERH of the compositions is maintained by mode of packing the compositions of the invention.

In an embodiment the present invention includes the pharmaceutical compositions comprising montelukast or its salts, wherein the compositions comprise less than about 2% or less than about 1% or less than about 0.5% of the mok-3 sulphoxide impurity.

In an embodiment the present invention includes the pharmaceutical compositions comprising montelukast or its salts, wherein the compositions comprise less than about 2% or less than about 1% or less than about 0.5% of the styrene impurity.

In another embodiment the present invention includes particle size of montelukast or its salt, wherein D₉₀ is not more than 250 μm or not more than 200 μm; D₅₀ is not more than 150 μm or not more than 100 μm; D₁₀ is not more than 100 μm or not more than 50 μm; D_{[4, 3]} is not more than 200 μm or not more than 150 μm or not more than 100 μm.

An embodiment the present invention includes the bulk density of montelukast or its salt which is in the range of about 0.2 g/ml to about 0.4 g/ml and tapped density which is in the range of about 0.4 g/ml to about 0.7 g/ml.

In another embodiment, the present invention further includes the particle size distribution of final blend for compression, wherein D₉₀ is in the range of about 400-850 μm.

In an embodiment the present invention includes bulk densities of final blend for compression in the range of about 0.2 g/ml to about 0.5 g/ml and tapped densities in the range of about 0.3 g/ml to about 0.7 g/ml.

In an embodiment the present invention further includes methods of using the pharmaceutical compositions in the treatment of asthma and allergic or perennial rhinitis.

### DETAILED DESCRIPTION

The present invention relates to pharmaceutical compositions of montelukast or their pharmaceutically acceptable salts, solvates, polymorphs, enantiomers or mixtures.

The number of people suffering from allergy-related disorders such as for example hay fever, allergic rhinitis, poison ivy contact, and asthma has increased in recent years.

The greatest prevalence of asthma is in preschool children. Asthma requires immediate perceivable effect. Inhaled therapy is most common therapy prescribed for young children. However, inhaled therapy has the disadvantage that dose delivery may be variable. Solid oral dosage form has the advantage of overcoming dose variability in comparison to inhaler therapy.

There are few cases where oral administration may not be possible such as when a patient has undergone surgery at neck portion (pharynx, esophageal, thyroid etc), and cannot swallow the solid dosage form. In such cases chewable dosage forms are used.

The pharmaceutically acceptable salts of montelukast refer to salts prepared form pharmaceutically acceptable non-toxic bases including inorganic bases and organic bases, or acids including inorganic and organic acids. Salts derived from inorganic bases include aluminum, ammonium, calcium, copper, ferric, ferrous, lithium, magnesium, manganic salts, manganous, potassium, sodium, zinc, and the like. Salts derived from organic non-toxic bases include, salts of primary, secondary, and tertiary amines, substituted amines including naturally occurring substituted amines.

Surprisingly it has been observed that the levels of two major impurities, namely mok-3 sulphoxide and a styrene impurity) increase due to presence of moisture content in the dosage form. Hence by reducing the moisture content of dosage forms by maintaining equilibrium relative humidity below a defined value, the levels of these impurities have been maintained below accepted levels.

During the manufacturing process, montelukast sodium is prone to a number of reactions which give rise to impurities. Some of the impurities, which may be generated as a result of the manufacturing process, include the following:

1) “Mok-3 sulphoxide” refers to 2-(1-{(1R)-1-{3-[(E)-2-(7-chloro-2-quinolyl)-1-ethenyl]phenyl}-3-[[2-(1-hydroxy-1-methyl ethyl)phenyl]propyl sulfinyl methyl}cyclopropyl]acetic acid, represented by Formula II.

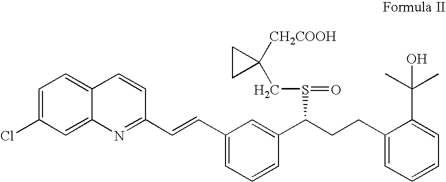

2) “QUID-8” refers to 2-[2-[3-(S)-[3-[2-[7-chloro-2-quinolinyl]ethenyl]phenyl]-3-hydroxy propyl]phenyl]-2-propanol, represented by Formula III.

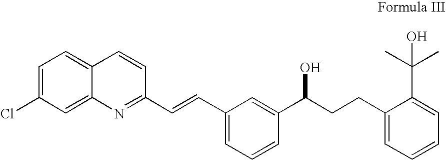

3) “Saturated analogue of montelukast” refers to 1-[[[(1R)-1-[3-[2-(7-chloro-2-quinolinyl)ethyl]phenyl]-3-[2-(1-hydroxyl-1-methylethyl)phenyl]propyl]thio]methyl]cyclopropane acetic acid, represented by Formula IV.

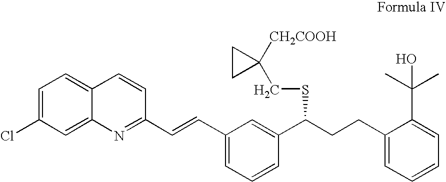

4) “Mok-3 keto” refers to 2-[1-(3-(2-acetylphenyl)-1-{3-[(E)-2-(7-chloro-2-quinolyl)-1-ethenyl]phenyl}propyl thio methyl)cyclopropyl]acetic acid, represented by Formula V.

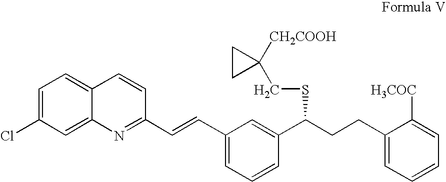

5) “Mok-1 nitrile” refers 1-[[[(1R)-1-[3-{(1E)-2-(7-chloro-2-quinolinyl)ethenyl]phenyl]-3-[2-(1-hydroxyl-1-methyl ethyl)phenyl]propyl]thio]methyl]cyclopropane acetonitrile, represented by Formula VI.

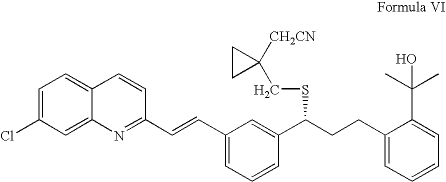

6) “Styrene impurity” refers to [R-(E)]-1-[[[1-[3-[2-(7-chloro-2-quinolinyl)ethenyl]phenyl]-3-[2-[1-(1-methyl)ethenyl)]phenyl]propyl]thio]methyl]cyclopropaneacetic acid, represented by Formula VIII.

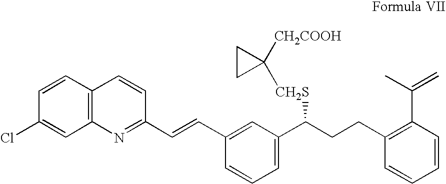

7) “S-isomer” refers to sodium salt of 1-[[[(1S)-1-[3-[(1E)-2-(7-chloro-2-quinolinyl) ethenyl]phenyl]-3-[2-(1-hydroxy-1-methyl ethyl)phenyl]propyl]thio]methyl]cycloproane acetic acid, represented by Formula VIII.

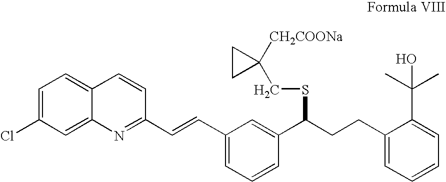

8) “Des-chloro impurity” refers to [R-(E)]-1-[[[1-[3-[2-(2-quinolinyl)ethenyl]phenyl]-3-[2-(1-hydroxy-1-methylethyl)phenyl]propyl]thio]methyl]cyclopropane acetic acid, represented by Formula IX.

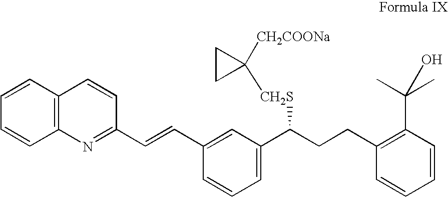

It has been observed that montelukast sodium is highly susceptible to oxidative degradation (including atmospheric oxygen), and acid hydrolysis with significant increase in the mok-3 sulfoxide and styrene impurities, respectively, thus mok-3 sulfoxide and styrene impurity are two of the major impurities.

Montelukast having a reduced level of impurities may also contain residual solvents such as hexane, cyclohexane, heptane, acetone, methanol, dichloromethane, acetonitrile, toluene, N,N-dimethylformamide, isobutyl alcohol. Thus, in an embodiment of the present invention, any residual solvents in purified montelukast are also considered as impurities.

In an embodiment of the present invention, the residual solvent content in the pharmaceutical compositions is less than the limits set in ICH guidelines.

In an embodiment the present invention includes stable pharmaceutical compositions.

In another embodiment the present invention includes the method of stabilizing the pharmaceutical compositions, which method include maintaining equilibrium relative humidity conditions for low inbuilt moisture during process of preparing the composition.

Relative humidity is defined as the ratio of the partial pressure of water vapor in a gaseous mixture of air and water to the saturated vapor pressure of water at a given temperature. Relative humidity is expressed as a percentage and is calculated in the following manner:

$$R H = \frac{{p}_{( {H}_{2} O )}}{{p}_{( {H}_{2} O )}^{*}} \times 100 \%$$

Equilibrium relative humidity or the ERH of a material is the relative humidity when the movement of moisture from a material to the environment (and vice versa) have equalized. This ERH balance is achieved when vapour pressures (within the material and in the environment) have equalized. At this point the moisture level of a material can be expressed in terms of ERH.

In an embodiment the present invention includes processes to achieve a desired value of ERH, wherein a process comprises:

a) placing desiccant and dosage forms in a triple laminated aluminum foil pouch; and

b) sealing the triple laminated aluminum foil pouch with desiccant arranged around the dosage forms.

Other sealed containers that have a high resistance to moisture ingress are also useful. Some of the containers of this kind include but are not limited to triple laminated pouches like triple laminated aluminium foil pouches, and high density polyethylene containers.

Suitable desiccants include, but are not limited to, silica gel, calcium sulfate, calcium chloride, montmorillonite clay, and molecular sieves.

In an embodiment the present invention includes pharmaceutical compositions containing montelukast, wherein the ERH for said pharmaceutical compositions is less than about 25%, 20%, 15%, or 10%.

Equilibrium relative humidity may be measured using instruments such as Beckman Hygroline Moisture Meter, Nova Sina/Rotronic Moisture-Humidity Meters, Hygrodynamic Hygrometer, and Weather-Measure Relative Humidity System. A discussion of their use in food analysis dated Apr. 16, 1984 has been provided by the U.S. Food and Drug Administration as “ITG SUBJECT: WATER ACTIVITY (a w) IN FOODS,” available at the URL address: http://www.fda.gov/ora/Inspect_ref/itg/itg39.html.

In an embodiment the present invention includes pharmaceutical compositions comprising montelukast or its salts, wherein said composition comprises less than about 2%, or less than about 1%, or less than about 0.5%, of the mok-3 sulphoxide impurity.

In an embodiment the present invention includes pharmaceutical compositions comprising montelukast or its salts, wherein said composition comprises less than about 1%, or less than about 0.5%, of the styrene impurity.

In an embodiment the present invention includes pharmaceutical compositions comprising montelukast or its salts, wherein said composition comprises less than about 1%, or less than about 0.5%, or less than about 0.1%, of the quid-8 impurity.

In an embodiment the present invention includes pharmaceutical compositions comprising montelukast or its salts, wherein said composition comprises less than about 1%, or less than about 0.5%, or less than about 0.1%, of the saturated analogue impurity.

In an embodiment the present invention includes pharmaceutical compositions comprising montelukast or its salts, wherein said composition comprises less than about 1%, or less than about 0.5%, or less than about 0.1%, of the mok-1 nitrile impurity.

In an embodiment the present invention includes pharmaceutical compositions comprising montelukast or its salts, wherein said composition comprises less than about 1%, or less than about 0.5%, or less than about 0.1%, of the mok-3 keto impurity.

In an embodiment the present invention includes pharmaceutical compositions comprising montelukast or its salts, wherein said composition comprises less than about 1%, or less than about 0.5%, or less than about 0.1%, of the deschloro impurity.

Various parameters impacting the compression process include the physical parameters of active as well as that of final blend as well as the compactability, flow, and other properties such as moisture content (determined by Karl Fischer (KF) apparatus or infra red moisture balance), particle size (determined by sieve analyzer or Malvern particle size analyzer), bulk density and tapped density, compressibility index, Hausner ratio (determined by USP density apparatus, flow property (determined by Flowdex apparatus) etc.

When a potent drug such as montelukast is present in a low concentration in the total composition, it is necessary to ensure that the active is uniformly distributed in the formulation so that there is no variation in the dose that is administered in unit dosage form. The uniformity of content of active is determined in terms of relative standard deviation.

The uniform distribution of the drug in the formulation may be achieved by many ways such as by using drug with uniform particle size distribution or by optimizing different steps of processing of the composition such as mixing and blending the active and inactive excipients or by selection of excipients and so on.

In an embodiment the present invention provides pharmaceutical compositions comprising montelukast or its salts, wherein said composition has uniformity of content of montelukast such that the relative standard deviation is not more than 6.

The particle size of a material may generally be described in terms of D₁₀, D₅₀, D₉₀, and D_{[4, 3]} used routinely to describe the particle size or size distribution. It is expressed as volume or weight or surface percentage. Dₓ as used herein is defined as the size of particles where x volume or weight percent of the particles have sizes less than the value given. D_{[4, 3]} for example is the volume mean diameter of the montelukast or the final blend for compression. D₉₀ for example means that 90% of the particles are below a particle size. Particle size or particle size distribution of the montelukast or final blend for compression of present invention are determined by the techniques that are known to the person skilled in the art including but not limited to sieve analysis, size analysis by laser principle such as Malvern particle size analyzer and the like.

In one of the embodiments the present invention, provides particle size distribution of the montelukast or its salt wherein: D₉₀ is not more than 250 μm, or not more than 200 μm; D₅₀ is not more than 150 μm, or not more than 100 μm; D₁₀ is not more than 100 μm, or not more than 50 μm; and D[₄, 3] is not more than 200 μm, or not more than 150 μm, or not more than 100 μm.

Another physicochemical characteristic of compositions is the density properties such as bulk and tapped density. Bulk density is described as untapped or tapped. Untapped bulk density of a substance is the undisturbed packing density of that substance and tapped bulk density relates to the packing density after tapping a bed of substance until no change in the packing density is seen. Bulk density and tapped density can be determined using a compendial bulk density apparatus, a suitable method being given in United States Pharmacopeia 29, United States Pharmacopeial Convention, Inc., Rockville, Md., 2005, at pages 2638-2639).

In an embodiment the present invention provides bulk density of montelukast or its salt in the range of about 0.2 g/ml to about 0.4 g/ml, and tapped density in the range of about 0.4 g/ml to about 0.7 g/ml.

In an embodiment, the present invention provides particle size distributions of final blends with excipients for compression where D₉₀ is in the range of from about 400 to 850 μm.

In an embodiment the present invention provides montelukast or its salts, wherein the bulk densities of montelukast or its salt range from about 0.2 g/ml to about 0.4 g/ml, and the tapped densities ranges from about 0.4 g/ml to about 0.7 g/ml.

In an embodiment the present invention provides pharmaceutical compositions comprising montelukast or its salts, wherein the bulk densities of the final blends with excipients for compression range from about 0.2 g/ml to about 0.5 g/ml, and the tapped densities range from about 0.3 g/ml to about 0.7 g/ml.

In an embodiment, the present invention provides pharmaceutical compositions comprising montelukast or its pharmaceutically acceptable salts, wherein said compositions have a moisture content of less than about 8%, about 5%, or about 3%, w/w.

In an embodiment the present invention provides pharmaceutical compositions comprising montelukast or its salts, wherein the compositions are solid oral dosage forms, such as tablets, capsules, lozenges, or pills. In yet another embodiment the present invention includes chewable solid dosage forms.

The solid dosage forms may include excipients, including but not limited to any one or more of fillers, binders, disintegrants, coloring agents, lubricating agents, glidants, sweeteners, flavorings and flavor enhancer agents, taste-masking agents, preservatives, buffers, wetting agents, coloring agents, and film-forming agents.

### Diluents:

Various useful fillers or diluents include but are not limited to starches, lactose, mannitol (Pearlitol SD200), cellulose derivatives, confectioners sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches included but not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, PH-112 microcrystalline cellulose 114, and microcrystalline cellulose 112. Other useful diluents include but are not limited to carmellose, sugar alcohols such as mannitol (Pearlitol SD200), sorbitol and xylitol, calcium carbonate, magnesium carbonate, dibasic calcium phosphate, and tribasic calcium phosphate.

### Binders:

Various useful binders include but are not limited to hydroxypropylcelluloses (Klucel™-LF), hydroxypropylcelluloses (Klucel EXF) hydroxypropyl methylcelluloses or hypromelloses (Methocel™), polyvinylpyrrolidones or povidones (PVP-K25, PVP-K29, PVP-K30, PVP-K90), Plasdone™ S 630 (copovidone), powdered acacia, gelatin, guar gum, carbomer (e.g. Carbopol™), methylcelluloses, polymethacrylates, and starches.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium(Ac-di-sol, FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropyl celluloses. Examples of low-substituted hydroxypropylcelluloses include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

### Coloring Agents:

Coloring agents can be used to color code the composition, for example, to indicate the type and dosage of the therapeutic agent therein. Suitable coloring agents include, without limitation, natural and/or artificial compounds such as FD & C coloring agents, natural juice concentrates, pigments such as titanium oxide, iron oxides, silicon dioxide, and zinc oxide, combinations thereof, and the like.

### Sweeteners:

Useful sweeteners include, but are not limited to: sugars such as sucrose, glucose (corn syrup), dextrose, invert sugar, fructose, and mixtures thereof; acid saccharin and its various salts such as the sodium or calcium salt; cyclamic acid and its various salts such as the sodium salt; the dipeptide sweeteners such as aspartame and alitame; natural sweeteners such as dihydrochalcone compounds; glycyrrhizin; Stevia rebaudiana (Stevioside); sugar alcohols such as sorbitol, sorbitol syrup, mannitol (PearlitolSD200), xylitol and the like, synthetic sweeteners such as acesulfame-K and sodium and calcium salts thereof and other synthetic sweeteners, hydrogenated starch hydrolysate (lycasin); protein based sweetening agents such as talin (thaumaoccous danielli); and/or any other pharmacologically acceptable sweetener, and mixtures thereof.

Suitable sugar alcohols useful as sweeteners include, but are not limited to, sorbitol, xylitol, mannitol (Pearlitol™ SD200), galactitol, maltitol, isomalt (PALATINIT™) and mixtures thereof. The exact amount of sugar alcohol employed is a matter subject to such factors as the degree of cooling effect desired.

### Flavoring Agents:

Flavoring agents can be used to improve the palatability of a composition. Examples of suitable flavoring agents include, without limitation, natural and/or synthetic (i.e., artificial) compounds such as peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (e.g. white, milk, dark), vanilla, caramel, coffee, hazelnut, combinations thereof, and the like.

### Lubricants:

An effective amount of any generally accepted pharmaceutical tableting lubricant can be added to assist with compressing tablets. Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

### Glidants:

One or more glidant materials, which improve the flow of the powder blends and minimizes the dosage form weight variation, can be used. Useful glidants include but are not limited to silicone dioxide, talc and combinations thereof.

### Solvents:

Solvents that are usegul during processing include but are not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethyl sulphoxide, dimethyl formamide, tetrahydrofuran, and mixtures thereof.

The final formulations may be coated or uncoated. For coating, additional excipients such as film-forming polymers, plasticizers, antiadherents and opacifiers are used.

### Film-Forming Agents:

Various film forming agents include but are not limited to cellulose derivatives such as soluble alkyl- or hydroalkylcellulose derivatives such as methylcellulose, hydroxymethyl cellulose, hydroxyethyl cellulose, hydroxypropyl cellulose, hydroxymethyethyl cellulose, hydroxypropyl methylcellulose, sodium carboxymethyl cellulose, etc., acidic cellulose derivatives such as cellulose acetate phthalate, cellulose acetate trimellitate, and methylhydroxypropylcellulose phthalate, polyvinyl acetate phthalate, etc., insoluble cellulose derivative such as ethylcellulose and the like, dextrins, starches and starch derivatives, polymers based on carbohydrates and derivatives thereof, natural gums such as gum Arabic, xanthans, alginates, polyacrylic acid, polyvinyl alcohol, polyvinyl acetate, polyvinylpyrrolidone, polymethacrylates such as derivatives thereof (Eudragit™), chitosan and derivatives thereof, shellac and derivatives thereof, waxes and fat substances.

If desired, the films may contain additional adjuvants for coating processing such as plasticizers, polishing agents, colorants, pigments, antifoam agents, opacifiers, antisticking agents, and the like.

As an alternative to the above coating ingredients, pre-formulated coating products such as Opadry™ Brown 03B86854 (supplied by Colorcon Limited, USA) may be used. The products that are sold in dry form require only mixing with a liquid before use.

In embodiments of the present invention, equipment suitable for processing the pharmaceutical compositions include mechanical sifters, granulators, blenders, roller compacter, compression machine, rotating bowls or coating pans, fluid bed processors, etc.

In an embodiment of the present invention, the pharmaceutical compositions may be processed by direct compression, dry granulation, or wet granulation.

In embodiments the present invention provides pharmaceutical compositions comprising montelukast or its salts in modified release dosage forms.

In aspects the present invention provides processes for preparing pharmaceutical compositions comprising montelukast or its salts, wherein an embodiment of a process comprises:

a) sifting the active ingredient through a sieve;

b) sifting excipients through a sieve;

c) mixing the sifted materials;

d) optionally granulating the dry mix from step c) using water, solvent, or a granulating solution or dispersion prepared by dissolving or dispersing binder in a suitable solvent;

e) drying the granules;

f) sifting the dried granules through a sieve;

g) adding sifted extragranular excipients to the sifted dried granules and blending;

h) adding a sifted lubricant to the blend of step g) and blending;

i) compressing the final lubricated blend into tablets or filling into empty hard gelatin capsule shells or into sachets; and

j) optionally coating tablets with a coating solution.

In an embodiment the invention includes processes for preparing the pharmaceutical compositions wherein the temperature during processing is not more than about 40° C. and the relative humidity is not more than about 60%, or the temperature during processing is not more than about 30° C. and the relative humidity is not more than about 50%.

The dosage forms prepared by the above process can be tested for physical parameters such as weight variation, hardness, disintegration test, friability etc. Several devices can be used to test tablet hardness such as a Monsanto tester, a Strong-Cobb tester, a Pfizer tester, a Erweka tester, a Schleuniger tester, etc. Friability can be determined using a Roche friabilator for 100 revolutions at 25 rpm. Disintegration time testing for tablets can be performed in a USP tablet disintegration tester wherein a tablet is placed in a basket, which moves upward and downward in a 1 L beaker of water at 37° C.

The tablets prepared by the above process can be subjected to in vitro dissolution evaluations according to Test 711 “Dissolution” in United States Pharmacopoeia 29, United States Pharmacopeial Convention, Inc., Rockville, Md., 2005 (“USP”) to determine the rate at which the active substance is released from the dosage forms, and the content of the active substance can be determined in solutions by high performance liquid chromatography.

In an embodiment the present invention includes the use of packaging materials such as containers and lids of high-density polyethylene (HDPE), low-density polyethylene (LDPE) and or polypropylene and/or glass, and blisters or strips composed of aluminium or high-density polypropylene, polyvinyl chloride, polyvinylidene dichloride, and aluminum/aluminium blisters with a laminated desiccant system. In another embodiment of the present invention, the packaging may also comprise various desiccants such as silica gel bags, molecular sieves, etc., which will enable the compositions to maintain the desired ERH levels.

In another embodiment the present invention provides pharmaceutical compositions comprising montelukast or its salts, wherein the percentages of montelukast in the total compositions are in the range of from about 1% to about 10% w/w.

In embodiments, a pharmaceutical formulation containing 10 mg of montelukast and administered orally in a single dose to healthy humans produces montelukast Cₘₐₓ values about 300 ng/mL to about 470 ng/mL, AUC₀₋₁ values about 2000 ng·hour/mL to about 3300 ng·hour/mL, and AUC_{0-∞} values about 2150 ng·hour/mL to about 3400 ng·hour/mL, in plasma.

In embodiments, a pharmaceutical formulation containing 5 mg of montelukast and administered orally in a single dose to healthy humans produces montelukast Cₘₐₓ values about 210 ng/mL to about 340 ng/mL, AUC₀₋₁ values about 1250 ng-hour/mL to about 2000 ng·hour/mL, and AUC_{0-∞} values about 13004 ng·hour/mL to about 2100 ng·hour/mL, in plasma.

In an embodiment the invention relates to analytical methods for analysis of impurities using high performance liquid chromatography (HPLC), wherein a method comprises:

Buffer Solution:

a) 5.2 g of dipotassium hydrogen phosphate (anhydrous) and 1 g of sodium perchlorate are dissolved in 1000 ml of Milli-Q water, pH is adjusted to 5 with orthophosphoric acid, and the solution filtered through a 0.45 μm Durapore hydrophilic membrane filter.

Mobile Phase A:

Buffer solution and acetonitrile are mixed in the volume ratio of 7:3.

Mobile Phase B:

Buffer solution and acetonitrile are mixed in the volume ratio of 3:7.

Diluent:

Milli-Q water and acetonitrile are mixed in the volume ratio of 4:6.

Chromatographic System:

a) The liquid chromatograph is equipped with a 225 nm UV detector.

b) Column: 4.6 mm×100 mm, 3 μm, Hypersil BDS-C18.

c) Column temperature: 30° C.

d) Flow rate: 1 ml per minute.

e) Injection volume: 20 μl.

f) Run time: 85 minutes.

The relative retention times of various impurities are tabulated below.

| Impurity | RRT* |
|---|---|
| Mok-3 | 0.32 and 0.36 |
| sulphoxide | |
| Quid -8 | 0.65 |
| Saturated | 0.83 |
| analogue | |
| Mok-3-keto | 1.04 |
| Mok-1-nitrile | 1.29 |
| Styrene impurity | 1.52 |

*Relative retention time, where montelukast = 1.

Certain specific aspects and embodiments of the invention are described in further detail by the examples below, which examples are provided solely for purposes of illustration and should not be construed as limiting the scope of the invention in any manner.

### EXAMPLES

### Example 1

### Pharmaceutical Compositions for Montelukast 10 mg Tablets

| Ingredient | mg/Tablet |
|---|---|
| Montelukast sodium | 10.4 |
| Lactose monohydrate | 89.3 |
| Microcrystalline cellulose (Avicel PH | 69.3 |
| 101)* | |
| Croscarmellose sodium (Ac-di-sol)** | 3 |
| Hydroxypropyl cellulose (Klucel-LF)# | 4 |
| Water‡ | 66 |
| Microcrystalline cellulose (Avicel PH | 20 |
| 112)* | |
| Croscarmellose sodium (Ac-di-sol) | 3 |
| Magnesium stearate | 1 |
| Opadry Brown 03B86854@ | 6 |
| Water‡ | 60 |

*Avicel PH 101 and Avicel PH 112 are supplied by FMC Biopolymer, USA
**Ac-di-sol is supplied by FMC Biopolymer, USA.
#Klucel-LF supplied by Aqualon.
@Opadry Brown 03B86854 is a mixture of hydroxypropyl methylcellulose 6 cps, titanium dioxide, polyethylene glycol, iron oxide yellow, iron oxide red, iron oxide black and is supplied by Colorcon Ltd.
‡Evaporates during processing.

Manufacturing Process:

1) Montelukast sodium and lactose monohydrate were co sifted in a 1:1 ratio through an ASTM #60 mesh sieve followed by sifting the remaining quantity of lactose monohydrate.

2) Materials from step 1), Avicel PH101, and Ac-di-sol were geometrically co-sifted through an ASTM #40 mesh sieve, then the mixture was sifted through an ASTM #40 mesh sieve.

3) Klucel LF was dissolved in water to form a granulating solution.

4) The mixture from step 2) was granulated with granulating solution from step 3), using a fluidized bed processor with a top spray granulation process having the following parameters:

Inlet temperature: 55° C.-60° C.

Blower rpm: 700-1200.

Spray pump rpm: 7-12.

The granules were dried at about 55° C. to about 60° C. until the loss on drying obtained was below about 2% w/w at 105° C. and the dried granules were sifted through a #30 mesh sieve.

5) The sifted granules from step 4) were placed into a 5 L double cone blender, Avicel PH 112 and Ac di sol, sifted through ASTM #40 mesh sieve, were added and the mixture was blended for about 15 minutes at about 20 rpm.

6) Magnesium stearate was sifted through an ASTM #40 mesh sieve, added to the step 5) mixture and blended for about 5 minutes.

7) The lubricated blend of step 6) was compressed into tablets.

8) Opadry Brown was dispersed in water and stirred well for about 45 minutes.

10) The tablets of Step 7) were coated using the coating suspension of step 8).

The above-prepared tablets were subjected to dissolution testing with 900 ml of 0.5% SLS in purified water as the medium, stirred at 50 RPM in USP II (Paddle) apparatus. Reference: SINGULAIR® 10 mg IR tablets. The data are in Table 1.

TABLE 1

Cumulative % of Drug
Dissolved

| Time | SINGULAIR ® | Example |
|---|---|---|
| (minutes) | 10 mg | 1 |
| 5 | 59 | 87 |
| 10 | 95 | 95 |
| 20 | 97 | 99 |
| 30 | 98 | 100 |

Tablets were packaged in sealed polyethylene bags containing six molecular sieve desiccant pouches (3 at the top and 3 at the bottom of the bags). The sealed bags were stored inside a triple laminated aluminum foil pouch for a minimum of 3 days prior to the final packaging. Equilibrium relative humidity of tablets after storing for about 3 days was 7.5% at the top and 10.6% at the bottom of bags. Tablets were then packaged individually in aluminum/aluminum blisters lined with silica gel as a desiccant, and stored at 40° C. and 75% RH conditions for 3 months. Then the compositions were analyzed for impurities (expressed as % of the montelukast content), moisture content (by KF), dissolution (30 minutes immersion in 0.5% sodium lauryl sulphate in 900 mL of purified water, 50 RPM stirring, USP apparatus 11), giving the data in Table 2.

TABLE 2

| | SINGULAIR ® 10 mg | | Example 1 | |
|---|---|---|---|---|
| Parameter | Initial | 3 Months | Initial | 3 Months |
| Mok-3- | 0.19 | 0.32 | 0.01 | 0.14 |
| sulphoxide | | | | |
| Saturated | 0.005 | 0.006 | 0.02 | 0.01 |
| analogue | | | | |
| Mok 3 keto | 0.01 | 0.014 | ND | ND |
| Styrene | ND | 0.103 | 0.03 | 0.04 |
| Total Impurities | 0.55 | 0.56 | 0.28 | 0.42 |
| Moisture | 4.13 | 4.54 | 4.5 | 5.3 |
| content (%) | | | | |
| Dissolution (%) | 98 | 99 | 100 | 87 |

ND = Not detected.

### Example 2

### Pharmacokinetic Parameters of Tablets Prepared According to Example 1

Tablets were evaluated in an open label, balanced, randomized two treatment, two-sequence, two period, two way crossover, single dose comparative bioavailability study with administration of the test product and the commercial product SINGULAIR® 10 mg tablets to 48 fasting healthy human volunteers, and plasma concentrations were determined at intervals after dosing.

The following parameters were calculated:

AUC₀₋₁=Area under plasma concentration versus time curve, from time zero (drug administration) to the last measurable concentration.

AUC_{0-∞}=Area under the plasma concentration versus time curve, from time zero to infinity.

Cₘₐₓ=Maximum plasma concentration.

Tₘₐₓ=Time after dosing until the maximum measured plasma concentrations.

The pharmacokinetic parameters from the study were calculated and are summarized in Table 3.

TABLE 3

| | Example | SINGULAIR ® | |
|---|---|---|---|
| Parameters | 1 (“T”) | 10 mg (“R”) | 100 × (T ÷ R) |
| AUC₀₋ₜ (ng · hour/mL) | 2601 | 2716 | 96 |
| AUC_{0-∞} (ng · hour/mL) | 2715 | 2816 | 96 |
| Cₘₐₓ (ng/mL) | 372 | 402 | 93 |
| Tₘₐₓ (hours) | 3.4 | 3.5 | — |

### Example 3

### Pharmaceutical Compositions for Montelukast 10 mg Tablets

| Ingredient | mg/Tablet |
|---|---|
| Montelukast sodium | 10.4 |
| Lactose monohydrate | 139.1 |
| Hydroxypropyl cellulose (Klucel-LF) | 6 |
| Water‡ | 0.11 |
| Croscarmellose sodium (Ac di sol) | 8 |
| Microcrystalline cellulose (Avicel PH 102) | 30.39 |
| Magnesium stearate | 1 |
| Opadry Brown 03B86854 | 5 |
| Water‡ | 50 |

‡Evaporates during processing.

Manufacturing process: similar to that of Example 1.

The tablets prepared in Example 3 were divided into two lots (A and B). Lot A tablets were stored at 25.5% ERH and Lot B tablets were subjected to reduction of ERH to 2% by packaging in polyethylene bags along with molecular sieve desiccant. Both Lot A and Lot B tablets were packaged in aluminum/aluminum blisters and stored for stability testing at 40° C. and 75% RH for 3 months. A commercial reference product SINGULAIR 10 mg tablets was similarly packaged and stored. The tablets were analyzed for impurities (expressed as % of montelukast content) and the data are tabulated in Table 4.

TABLE 4

| | | | 1 Month | | | 3 Months | | |
|---|---|---|---|---|---|---|---|---|
| | Initial | | Example 3 | | | Example 3 | | |
| Impurity | Reference | Example 3 | Lot A | Lot B | Reference | Lot A | Lot B | Reference |
| Mok-3 | 0.19 | 0.09 | 0.39 | 0.1 | 0.32 | 0.66 | 0.08 | 0.32 |
| sulphoxide | | | | | | | | |
| Styrene | ND | 0.04 | 0.04 | 0.03 | 0.12 | ND | 0.02 | 0.1 |
| Total | 0.55 | 0.57 | 0.84 | 0.44 | 0.54 | 1.46 | 0.7 | 0.56 |
| Impurities | | | | | | | | |

ND = not detected.

### Example 4

### Pharmaceutical Compositions for Montelukast 10 mg Tablets Prepared by Direct Compression

| Ingredient | mg/Tablet |
|---|---|
| Montelukast sodium | 10.4 |
| Lactose monohydrate (Tablettose 80)* | 90.1 |
| Lactose monohydrate | 20 |
| Microcrystalline cellulose (Avicel PH 102) | 77 |
| Croscarmellose sodium (Ac-di-sol) | 10 |
| Magnesium stearate | 1.5 |
| Opadry Brown 03B86854 | 5 |
| Water‡ | 50 |

*Tablettose 80 is supplied by Meggle Pharma, Germany.
‡Evaporates during processing.

Manufacturing Process:

1) Montelukast sodium and lactose monohydrate were co-sifted geometrically through a #60 mesh sieve.

2) Tablettose 80, Avicel PH102 and Ac-di-sol were sifted through an ASTM #40 mesh sieve.

3) The sifted materials of step 1 and step 2 were mixed well for about 4 minutes.

4) Magnesium stearate was sifted through an ASTM #40 mesh sieve, added to the mixture of step 3 and mixed well for about 2 minutes.

5) The lubricated blend of step 4 was compressed into tablets using 8×8 mm, rounded, square shaped punches.

Coating:

6) Opadry Brown was dispersed in water and stirred well for about 45 minutes.

7) The core tablets of step 5 were coated with the coating suspension prepared in step 6).

### Example 5

### Pharmaceutical Compositions for Montelukast 10 mg Tablets Prepared Using Non-Aqueous Granulation

| Ingredient | mg/Tablet |
|---|---|
| Montelukast sodium | 10.4 |
| Lactose monohydrate | 90.1 |
| Microcrystalline cellulose (Avicel PH 101) | 62 |
| Croscarmellose sodium (Ac-di-sol) | 5 |
| Hydroxypropyl cellulose (Klucel-LF) | 6 |
| Isopropyl alcohol (IPA) | 100 |
| Microcrystalline cellulose (Avicel PH 112) | 20 |
| Croscarmellose sodium (Ac-di-sol) | 5 |
| Magnesium stearate | 1.5 |
| Opadry Brown 03B86854 | 5 |
| Water‡ | 50 |

‡Evaporates during processing.

Manufacturing Process: Similar to that for Example 1.

### Example 6

### Pharmaceutical Compositions for Montelukast 5 mg Chewable Tablets

| Ingredient | mg/Tablet |
|---|---|
| Montelukast sodium | 5.2 |
| Mannitol | 201.35 |
| Hydroxypropyl cellulose (Klucel EXF*, part 1) | 7 |
| Croscarmellose sodium (Ac-di-sol, part I) | 4.5 |
| Hydroxypropyl cellulose (Klucel EXF, part II) | 2 |
| Iron oxide, red | 0.45 |
| Isopropyl alcohol‡ | 150 |
| Croscarmellose sodium (Ac-di-sol, part II) | 4.5 |
| Microcrystalline cellulose (Avicel PH 112) | 69.5 |
| Aspartame | 1.5 |
| Cherry flavor | 1 |
| Magnesium stearate | 3 |

‡Evaporates during processing.
*Klucel EXF is marketed by Aqualon.

Manufacturing Process:

1) Montelukast sodium and mannitol, were sifted through an ASTM #60 mesh sieve.

2) Klucel EXF part I and Ac-di-sol part I were sifted through an ASTM #40 mesh sieve.

3) Step 1 and 2 materials were cosifted through an ASTM #40 mesh sieve.

4) Isopropyl alcohol was divided into two parts. Klucel EXF part 11 was dissolved in isopropyl alcohol part I and stirred until it formed a clear solution.

5) Iron oxide red was sifted through an ASTM #80 mesh sieve and added to isopropyl alcohol part 11, with stirring for about 15 to 20 minutes.

6) Step 5) was added to step 4) with stirring for about 10-15 minutes.

7) Sifted materials of step 3) were loaded into fluid bed bowl.

8) Step 7) materials were granulated with an inlet temperature of 50-60° C. using the dispersion of step 6) as a top spray.

9) After granulation, the drying was continued until loss on drying at 105° C. was below 2% w/w.

10) Dried granules were sifted through an ASTM #25 mesh sieve.

11) Avicel PH 112, croscarmellose sodium part 11 and aspartame were sifted through an ASTM #40 mesh sieve.

12) Granules from step 10) and sifted materials from step 11) were blended for about 15 minutes.

13) Cherry flavour and magnesium stearate were sifted through an ASTM #60 mesh sieve, added to materials of step 12) and blended for about 5 minutes.

14) The lubricated blend from step 13) was compressed into tablets.

The tablets prepared were divided into two lots (lot C and lot D). Lot C tablets were stored in a sealed polyethylene bag with a molecular sieve desiccant to produce an ERH of 15% and lot D tablets were similarly stored to produce an ERH of 10%. Lot C and Lot D tablets, and SINGULAIR 5 mg tablets, were packaged in aluminum/aluminum blisters and stored for stability testing at 40° C. and 75% RH. Tablets were analyzed for their impurity contents (expressed as % of montelukast content) and the data are tabulated in Table 5.

TABLE 5

| | | | |
|---|---|---|---|
| Sample | Mok-3 sulphoxide | Styrene | Total impurities |

Example 6

| | | | |
|---|---|---|---|
| Initial | 0.06 | 0.02 | 0.67 |
| Lot C, 1 month | 0.024 | 0.007 | 0.29 |
| Lot C, 2 months | 0.33 | 0.02 | 0.71 |
| Lot D, 1 month | 0.01 | 0.025 | 0.44 |
| Lot D, 2 months | 0.13 | 0.025 | 0.54 |

SINGULAIR

| | | | |
|---|---|---|---|
| Initial | 0.3 | 0.09 | 0.73 |
| 1 month | 0.6 | ND | 1.164 |
| 2 months | 0.64 | 0.125 | 1.1 |

ND = Not detected.

### Examples 7-8

### Pharmaceutical Compositions for Montelukast 5 mg Chewable Tablets

mg/Tablet

| | | |
|---|---|---|
| Ingredient | Example 7 | Example 8 |

Intragranular

| | | |
|---|---|---|
| Montelukast sodium | 5.2 | 5.2 |
| Mannitol | 201.35 | 201.35 |
| Hydroxypropyl cellulose (Klucel | 7 | 7 |
| EXF, part I) | | |
| Croscarmellose sodium (Ac-di-sol) | 9 | 9 |

Binder Dispersion

| | | |
|---|---|---|
| Hydroxypropyl cellulose (Klucel | 2 | 2 |
| EXF, part II) | | |
| Iron oxide, red | 0.45 | 0.25 |
| Isopropyl alcohol‡ | 150 | 150 |

Extragranular

| | | |
|---|---|---|
| Microcrystalline cellulose (Avicel | 69.5 | 69.5 |
| PH 112) | | |
| Aspartame | 1.5 | 1.5 |
| Iron oxide, red | — | 0.2 |
| Cherry flavor | 1 | 1 |
| Magnesium stearate | 3 | 3 |

‡Evaporates during processing.

Manufacturing Process: Similar to that for Example 6.

The tablets prepared in Example 7 were packaged in sealed polyethylene bags containing 4 molecular sieve pouches (2 at the top and 2 at the bottom of the bags) as a desiccant. The sealed bags were stored inside a triple laminated aluminium foil pouch for minimum of 3 days prior to the final packaging. Equilibrium relative humidity of tablets after storing for about 3 days was 10.5% at the top and 11.6% at the bottom of the bags. Then these tablets were packaged in HDPE containers and stored for stability testing at 40° C. and 75% RH for 3 months.

Tablets prepared in Example 8 were packaged in aluminum/aluminum foil blisters, lined with desiccant. The tablets and SINGULAIR 5 mg chewable tablets were stored for 3 months at 40° C. and 75% RH. The impurities (expressed as % of the montelukast content), water content (by KF) and drug dissolution (30 minutes immersion in 0.5% sodium lauryl sulphate in 900 mL of purified water, 50 RPM stirring, USP apparatus II) were determined and analysis data are in Table 6.

TABLE 6

| | SINGULAIR 5 mg | | Example 7 | | Example 8 | |
|---|---|---|---|---|---|---|
| Parameter | Initial | 3 Months | Initial | 3 Months | Initial | 3 Months |
| MOK-3 Sulphoxide | 0.49 | 1.02 | 0.09 | 0.41 | 0.2 | 0.28 |
| Styrene impurity | 0.08 | 0.09 | 0.03 | 0.07 | 0.07 | 0.1 |
| Total Impurities | 0.75 | 1.29 | 0.48 | 0.71 | 0.58 | 0.61 |
| Water by KF (% | 1.4 | 1.73 | 2.68 | 1.7 | 1.5 | 0.8 |
| w/w) | | | | | | |
| Dissolution (%) | 99 | 88 | 97 | 97 | 97 | 90 |

The tablets of Examples 7 and 8 and SINGULAIR 5 mg chewable tablets were subjected to dissolution testing in 900 ml of 0.5% SLS in purified water, 50 RPM stirring, in USP II (paddle) apparatus. The cumulative percentages of drug dissolved are tabulated in Table 7:

TABLE 7

| Time (minutes) | SINGULAIR ® 5 mg | Example 6 | Example 7 |
|---|---|---|---|
| 5 | 82 | 82 | — |
| 10 | 91 | 89 | 94 |
| 20 | 93 | 91 | 97 |
| 30 | 93 | 92 | 98 |

Tablets prepared in Example 7 were evaluated in an open label, balanced, randomized two treatment, two-sequence, two period, two way crossover, single dose comparative bioavailability study with administration of the test product and the commercial product SINGULAIR® 5 mg to 48 fasting healthy human subjects, and plasma concentrations were determined at intervals after dosing.

The calculated pharmacokinetic parameters are summarized in Table 8.

TABLE 8

| | Example | SINGULAIR ® | |
|---|---|---|---|
| Parameter | 7 (“T”) | 5 mg (“R)” | 100 × (T ÷ R) |
| AUC₀₋ₜ (ng · hour/mL) | 1582 | 1717 | 92 |
| AUC_{0-∞} (ng · hour/mL) | 1683 | 1806 | 93 |
| Cₘₐₓ (ng/mL) | 270 | 284 | 95 |
| Tₘₐₓ (hours) | 2.94 | 2.83 | — |

### Examples 9-12

### Montelukast 5 mg Chewable Tablets by Aqueous Granulation

| | mg/Tablet | | | |
|---|---|---|---|---|
| | Example | Example | Example | Example |
| Ingredient | 9 | 10 | 11 | 12 |
| Montelukast sodium | 5.2 | 5.2 | 5.2 | 5.2 |
| Mannitol (impalpable)$ | 200 | 215.35 | 201.35 | 212.35 |
| Microcrystalline cellu- | 64.5 | 30 | — | 60 |
| lose (Avicel PH 101) | | | | |
| Croscarmellose sodium | — | 6 | 9 | — |
| (Ac-di-sol, part I) | | | | |
| Hydroxypropyl cellu- | 6 | — | 9 | 3 |
| lose (Klucel EXF) | | | | |
| Iron oxide, red | 0.45 | 0.45 | 0.45 | 0.45 |
| Water‡ | 0.06 | 0.18 | 75 | 117 |
| Croscarmellose sodium | 9 | 6 | — | 12 |
| (Ac-di-sol, part II) | | | | |
| Microcrystalline cellu- | | 30 | 69.5 | — |
| lose (Avicel PH 112) | | | | |
| Aspartame | 10 | 3 | 1.5 | 3 |
| Cherry flavour | 1 | 1 | 1 | 1 |
| Magnesium stearate | 3.85 | 3 | 3 | 3 |

‡Evaporates during processing.
$Mannitol (impalpable): supplied by Roquette

Manufacturing Process:

1) Montelukast sodium, mannitol and Avicel PH 101 were sifted through an ASTM #40 mesh and dry mixed for about 15 minutes.

2) Water was divided into two parts. Klucel EXF (in Example 10, there is no Klucel) was dissolved in water, part I with stirring. Iron oxide red was dispersed in water with stirring to form a uniform dispersion. Iron oxide dispersion was added to Klucel EXF solution to form granulating dispersion.

3) The dry mixture from step 1) was granulated using the granulating dispersion from step 2).

4) The granules from step 3) were dried at about 55° C. in fluid bed drier until the loss on drying at 105° C. was about 1-2% w/w.

5) The dried granules from step 4) were sifted through an ASTM #25 mesh sieve.

6) Croscarmellose sodium part 11 and aspartame were sifted through an ASTM #40 mesh sieve, added to the sifted granules of step 5 and blended for about 15 minutes.

7) Cherry flavor and magnesium stearate were sifted through an ASTM #60 mesh sieve, added to the blend of step 6) and blended for about 5 minutes.

8) The final lubricated blend of step 7) was compressed into tablets.

### Example 13

### Pharmaceutical Compositions for Montelukast 4 mg Chewable Tablets

| Ingredient | mg/Tablet |
|---|---|
| Montelukast sodium | 4.16 |
| Mannitol (impalpable) | 161.08 |
| Hydroxypropyl cellulose (Klucel EXF part I) | 5.6 |
| Croscarmellose sodium (Ac-di-sol) | 7.2 |
| Hydroxypropyl cellulose (Klucel EXF, part II) | 1.6 |
| Iron oxide, red | 0.2 |
| Isopropyl alcohol (IPA)‡ | 120 |
| Microcrystalline cellulose (Avicel PH 112) | 55.6 |
| Aspartame | 1.2 |
| Cherry flavour | 0.8 |
| Iron oxide, red | 0.16 |
| Magnesium stearate | 2.4 |

‡Evaporates during processing.

Manufacturing Procedure: Similar to that for Example 6.

The tablets were subjected to in vitro dissolution testing with the following parameters, and the data are tabulated in Table 8:

Medium: 0.5% Sodium lauryl sulphate in water.

Agitation: 50 RPM.

Apparatus: USP II (Paddle).

Volume: 900 ml.

Reference: SINGULAIR® 4 mg chewable tablets.

TABLE 8

Cumulative % of Drug Dissolved

| Time (minutes) | SINGULAIR ® 4 mg | Example 13 |
|---|---|---|
| 10 | 91 | 90 |
| 20 | 98 | 95 |
| 30 | 99 | 95 |
| 45 | 99 | 95 |

The tablets prepared according to Example 13 were packaged in sealed polyethylene bags containing 4 molecular sieve pouches (2 at the top and 2 at the bottom of the bags) as a desiccant. The sealed bags were stored inside a triple laminated aluminum foil pouch for minimum of 3 days prior to the final packaging. Equilibrium relative humidity of tablets after storing for about 3 days was 8.5% at the top and 13% at the bottom of the bags. Then these tablets were packaged in aluminum/aluminum foil blisters lined with desiccant and stored for stability testing at 40° C. and 75% RH for 3 months.

The impurities (expressed as % of montelukast content), water content (by KF) and drug dissolution (30 minutes immersion in 0.5% sodium lauryl sulphate in 900 mL of purified water, 50 RPM stirring, USP apparatus II) were determined and analysis data are in Table 10.

TABLE 10

Example 13

| Parameter | Initial | 3 Months |
|---|---|---|
| MOK-3 sulphoxide impurity | 0.2 | 0.29 |
| Styrene impurity | 0.07 | 0.08 |
| Total impurities | 0.65 | 0.58 |
| Water by KF (% w/w) | 1.4 | 1.2 |
| Dissolution (%) | 95 | 93 |

We claim:

1. A solid pharmaceutical formulation for oral administration comprising montelukast or a salt thereof, and at least one pharmaceutically acceptable excipient, wherein the formulation has an equilibrium relative humidity less than about 25 percent.

2. The solid pharmaceutical formulation of claim 1, wherein the equilibrium relative humidity is less than about 20 percent.

3. The solid pharmaceutical formulation of claim 1, wherein the equilibrium relative humidity is less than about 15 percent.

4. The solid pharmaceutical formulation of claim 1, wherein the equilibrium relative humidity is less than about 10 percent.

5. The solid pharmaceutical formulation of claim 1, having, after storage for 3 months at 40° C. and 75 percent relative humidity in a sealed package with a desiccant, less than about 2 percent by weight of an initial montelukast content of at least one of the impurities having the structures:

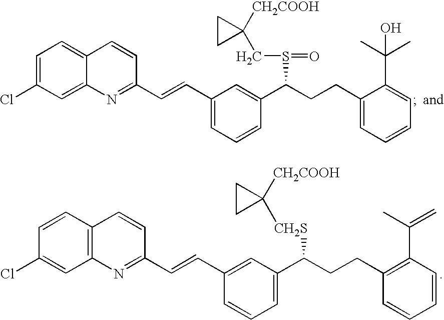

6. The solid pharmaceutical formulation of claim 5, having, after storage, less than about 2 percent by weight of an initial montelukast content of each of the impurities.

7. The solid pharmaceutical formulation of claim 5, having, after storage, less than about 1 percent by weight of an initial montelukast content of at least one of the impurities.

8. The solid pharmaceutical formulation of claim 7, having, after storage, less than about 1 percent by weight of an initial montelukast content of each of the impurities.

9. The solid pharmaceutical formulation of claim 5, having, after storage, less than about 0.5 percent by weight of an initial montelukast content of at least one of the impurities.

10. The solid pharmaceutical formulation of claim 9, having, after storage, less than about 0.5 percent by weight of an initial montelukast content of each of the impurities.

11. The solid pharmaceutical formulation of claim 1, containing less than about 8 percent by weight moisture.

12. The solid pharmaceutical formulation of claim 1, containing 10 mg of montelukast equivalent and producing montelukast Cₘₐₓ values about 300 ng/mL to about 470 ng/mL, AUC₀₋ₜ values about 2000 ng-hour/mL to about 3300 ng·hour/mL, and AUC_{0-∞} values about 2150 ng·hour/mL to about 3400 ng·hour/mL, in plasma after oral administration of a single dose to healthy humans.

13. The solid pharmaceutical formulation of claim 1, containing 5 mg of montelukast equivalent and producing montelukast Cₘₐₓ values about 210 ng/mL to about 340 ng/mL, AUC₀₋ₜ values about 1250 ng·hour/mL to about 2000 ng·hour/mL, and AUC_{0-∞} values about 1300 ng·hour/mL to about 2100 ng·hour/mL, in plasma after oral administration of a single dose to healthy humans.

14. A process for preparing a solid pharmaceutical formulation for oral administration comprising montelukast or a salt thereof, comprising reducing an equilibrium relative humidity level of a formulation below about 25 percent.

15. The process of claim 14, wherein an equilibrium relative humidity level is reduced below about 10 percent.

16. The process of claim 14, wherein an equilibrium relative humidity level is reduced by storing a solid pharmaceutical formulation in a sealed container with a desiccant.

17. The process of claim 16, wherein a sealed container comprises a laminated aluminum foil bag.

18. A solid pharmaceutical formulation prepared by the process of claim 14.

19. A solid pharmaceutical formulation prepared by the process of claim 15.

20. The solid pharmaceutical formulation prepared by the process of claim 14, containing less than about 8 percent by weight moisture.
